(19) United States
(12) Patent Application Publication
(10) Pub. No.: US 2013/0202564 A1
(43) Pub. Date: Aug. 8, 2013

(54) Systems and Methods of Cell Activated, Controlled Release Delivery of Growth Factors for Tissue Repair and Regeneration

(71) Applicants: Bo Han, Temple City, CA (US); Kenrick Kuwahara, Woodland Hills, CA (US)

(72) Inventors: Bo Han, Temple City, CA (US); Kenrick Kuwahara, Woodland Hills, CA (US)

(73) Assignee: UNIVERSITY OF SOUTHERN CALIFORNIA, Los Angeles, CA (US)

(21) Appl. No.: 13/640,027

(22) PCT Filed: Apr. 8, 2011
(86) PCT No.: PCT/US11/31860

§ 371 Date: Apr. 19, 2013

Related U.S. Application Data
(60) Provisional application No. 61/322,733, filed on Apr. 9, 2010.

(51) Int. Cl. A61K 47/42; A61K 38/54

(52) U.S. Cl. CPC A61K 47/42; A61K 38/54
USPC 424/93.7; 424/94.2; 424/94.3; 435/174; 435/177

(57) ABSTRACT

This invention provides a composition for controlled-release of polypeptide growth factors useful in tissue repair or engineering. The composition include a polypeptide growth factor covalently cross-linked to a biocompatible substrate by a transgultaminase. The cross-linking tethers the growth factor to a substrate so that it will not diffuse away from the desired site of application. It also concomitantly inactivates the growth factor. Release and reactivation of the growth factor can be achieved by endogenously produced metalloproteinase (MMPs) or exogenously provided proteases such as collagenases. Also provided are scaffolds, transplant devices, methods for using the same and methods for making the same,

### CROSS-REFERENCE TO RELATED APPLICATIONS

This application claims the benefit of U.S. Provisional Application No. 61/322,733, file on Apr. 9, 2010. The above application(s) is hereby incorporated herein by reference.

### FIELD OF THE INVENTION

The invention pertains to the field of tissue engineering. More particularly, the invention pertains to systems and methods for delivering growth factors to effect tissue repair and regeneration in a cell-activated and controlled-release manner

### BACKGROUND OF THE INVENTION

The following is offered as background information only and is not admitted to being prior art to the present invention.

In order for tissues to repair or regenerate, cells must first migrate into a wound bed, proliferate, express matrix components or form extracellular matrix, and then form a final tissue shape. This process involves a variety of cell populations interacting in an intricate web of cascading events. To facilitate this process, successful artificial regeneration of tissues usually requires a physical scaffold for cell migration and proliferation. However, most scaffolds do not work by themselves without the addition of growth factors.

Growth factors are signaling molecules that help cells differentiate and preserve the biological functions of the repaired tissues. For example, in bone repair and regeneration, bone morphogenetic proteins (BMPs) are the growth factors that promote and regulate bone formation. In particular, BMP-2 has the ability to regulate the differentiation of osteoblastic progenitor cells and the ability to transdifferentiate non-osteogenic cells towards an osteoblastic lineage in vitro. By recruiting progenitor cells, BMP-2 is capable of inducing new bone formation at ectopic and orthotropic sites. In clinical studies, BMP-2 has the potential to replace autografts by inducing new bone growth for spinal fusion and non-union bone healing. Although much is known about the biological functions of growth factors such as BMP-2, successful utilization of this knowledge in clinical applications of tissue engineering is still fraught with difficulties.

One major problem in using growth factors to induce tissue regeneration is that they must be present at certain concentrations at the site of regeneration. In many occasion, growth factors have a short half-life due to degradation or/and digestion and tend to disperse rapidly away from the targeted site. Dispersion of growth factors not only minimizes their effects, but may also induce undesirable side effects to the surrounding tissues. For example, in the case of bone regeneration, BMP-2 concentration in demineralized bone matrix is estimated to be 1-2 μg in a kg of bone. Currently, the typical amount of growth factors used in clinical applications to overcome the problems of short half-life and rapid dispersion is grossly above physiological concentrations. For example, in lumbar spine fusion, about 12-16 mg of BMP-2 per dose is used. This amount is million times higher than physiological dose, but is needed to ensure adequate biological activity. Such brute force approach is not costly but also poses significant risk of inducing undesirable side effects. For example, in a bone defect study by Liu et al., locally administered high dosages of BMP-2 have been shown to cause heterotopic bone formation and bone cyst formation. Overdose can also trigger negative feedback loop where BMP-2 inhibitors may be released.

Aside from bioavailability, the timing of growth factor release is another significant challenge. In the same study by Liu et al., bone formation was found to be impaired when bursts of BMP-2 was released in the early phase of healing. Thus, a delayed and sustained release of growth factors is more beneficial to tissue regeneration and can minimize the amount of growth factors needed.

Presently, the most common delivery system for BMPs involves a degradation/diffusion-based delivery scheme that utilizes the physical properties of the scaffold materials to regulate its availability. Scaffold material used include ceramics, nano/microparticles, biodegradable synthetic polymers and collagen. In most instances, the scaffolds are constructed in such a way that BMPs are non-covalently immobilized for the purpose of increasing retention time while maintaining an acceptable level of availability. This allows osteoprogentitor cells to migrate and differentiate into osteoblasts at the repair sites.

The major problem associated with this approach is that because of the non-covalent coupling between the BMPs and the scaffold materials, a high initial burst release of BMPs inevitably results. To compensate for this initial rapid depletion of BMPs, the BMPs are always applied in excessive concentrations which significantly increases the risk of undesirable side effects.

One approach to circumvent this problem is by modified the growth factors to enhancing their affinity for a particular substrate. For example, Han el al. have synthesized recombinant fusion proteins of TGF-β and BMP-3 that contain a collagen-binding domain derived from von Willebrand factor to allow for enhanced non-covalent immobilization on the surface of collagen-based materials (Han et al., J Orthop Res, 2002. 20(4): p. 747-55). However, this approach is far from ideal for clinical use because it requires production of fusion growth factors which is expensive, cumbersome, and may alter the bioactivity of the growth factors.

Another approach is to covalently tether BMPs to a substrate. Schmoekel et al. (Schmoekel et al., Biotechnol Bioeng, 2005. 89(3): p. 253-62) comes close to binding the BMP-2 covalently by creating a recombinant fusion protein consisting of BMP-2. While this approach affords a higher growth factor retention rate, it also suffers from the same shortcoming of altered efficacy or biological activity due to uncontrolled cross-linking or protein denaturing.

Similar to Schmoekel, U.S. Pat. No. 6,894,022 to Hubbell discloses growth factor-modified protein matrices for tissue engineering. In this system, the growth factors are incorporated into the matrices so that they are released by degradation of the matrices, enzymatic action, and/or diffusion. Hubbell's system requires the creation of fusion proteins that contains the growth factor fused to a heparin interacting domain in order to incorporate the growth factors into the matrices. It is also cumbersome and expensive to implement.

In yet another approach, substrate specific enzymatic cross-linking methods have been used to generate control delivery of growth factors. For example, ross-linking enzymes such as factor XIII or tissue transglutaminase (Ehrbar et al., Biomaterials, 2007. 28(26): p. 3856-66) and tyrosinase (Demolliens et al., Bioconjug Chem, 2008. 19(9): p. 1849-54) which selectively recognize certain amino acid sequences have been used to immobilize growth factors such as VEGF and NGF to matrices without compromising their activities. However, this approach also suffers from the drawback that growth factors must be modified or re-engineered to contain additional sequences that are compatible with the enzymatic reactions.

Therefore, there still exists a need for systems and methods that are simple and cost effective while still capable of delivering growth factors to the targeted site in a cell activated, controlled release manner.

### SUMMARY OF THE INVENTION

The present invention is based on the unexpected discovery that polypeptide growth factors can be cross-linked to a biocompatible carrier substrate by an enzymatic reaction, in particular, by a transglutaminase (TGase). More importantly, the cross-linked growth factor becomes inactivated upon being cross-linked to the substrate. The linkage is through a covalent bond between the growth factor and the substrate. No linker or other binding domain is required, allowing the growth factor to be delivered in its native form without other modifications such as addition of a linker or a fusion domain for binding. The de-activated growth factor can be reactivated by enzymatically digesting away the carrier substrate. This scheme greatly simplifying the application of the method.

Another even more surprising discovery is that protease or metalloproteinase may be used to reverse the cross-linking reaction. These enzymes are specific for the substrate but will not harm the polypeptide growth factors. When the covalent bond is broken by these enzymes, the polypeptide growth factor is released from the substrate and become active again. Fortuitously, metalloproteinase such as collagenase are secreted by cells. Thus, growth factors may be delivered to target cells in its inactive form and then activated automatically by the target cells.

These unexpected discoveries of the present invention form the basis for a very simple and elegant strategy for the storage and delivery of growth factor.

Accordingly, in a first aspect, the present invention provides a composition for delivery and controlled-release of a polypeptide growth factor. Embodiments in accordance with this aspect of the invention generally include a therapeutically effective amount of a polypeptide growth factor; and a biocompatible carrier substrate. The polypeptide growth factor is covalently cross-linked to the carrier substrate by an enzymatic linking agent that directly links the growth factor to the peptide carrier. Upon being cross-linked, the growth factor becomes reversibly inactivated.

As used herein, the term “therapeutically effective amount” means an amount sufficient to carry out a specifically stated therapeutic purpose. An “effective amount” may be determined empirically and in a routine manners in relation to the stated purpose.

The growth factor is generally in its native, unmodified form, meaning that it has its native peptide sequence without modification. Growth factors that may be used in a composition of the present invention are not particularly limited so long as it can be enzymatically cross-linked to a peptide carrier directly without the use of any linker. Suitable growth factors may include but not limited to BMP10, BMP15, BMP2, BMP3, BMP4, BMP5, BMP6, BMP7, BMP8A, BMP8B, TGFB1, TGFB2, TGFB3, GDF1, GDF10, GDF11, GDF15, GDF2, GDF3, GDF3A, GDF5, GDF6, GDF7, GDF8, GDF9, GDNF, INHA, INHBA, INHBB, INHBC, INHBE, LEFTY1, LEFTY2, MSTN, NODAL, NRTN, PSPN, AMH, and ARTN. Other growth factors useful for tissue repair and regeneration such as FGF, PDGF, VEGF, IGF, and NGF may also be used.

In a preferred embodiment, the growth factor is a bone morphogenetic protein, more preferably BMP-2.

Carrier substrate suitable for use in a composition of the present invention is also not particularly limited so long as it can be enzymatically cross-linked to the growth factor directly and can be selectively digested by an enzyme. Any biocompatible material having surface exposed lysine at a catalytic site recognizable by a TGase may be used as a carrier substrate. The carrier substrate may be in the form of a matrix or a free standing molecule. Exemplary carrier substrates may include gelatin, collagen, and albumin, fibrin, fibrinogen, laminin, fibronectin, vitronectin, a synthetic peptide containing an exposed lysine or glutamine, but are not limited thereto. Those skilled in the art will recognize that depending on the enzyme used, peptides containing certain sequences are more preferable than others. In some preferred embodiments, the peptide carrier is a collagen or a derivative thereof, more preferably a gelatin.

Suitable enzymatic linking agents is preferably a transglutaminase, more preferably a bacterial transgultaminase.

To release the growth factor from the delivery composition, an activating agent must be added. This activating agent may be exogenously added or may be secreted from a target where the growth factor is intended to be delivered, for example, a wound site, or a cell culture. Ideally, the activating agent is an enzyme specific for enzymatically breaking the covalent bond between the growth factor and the peptide carrier. It may enzymatically break down the carrier peptide, but should be harmless for the growth factor. Suitable activating agents may include proteases or metalloproteinases. Exemplary activating agents may include, but not limited to pronase, trypsin, chymopapain, chymotrypsin, papain, collagenase, plasmin, pepsin, elastase, MMP1, MMP2,MMP3, MMP8, MMP9, MMP10, MMP13, MMP14 and MMP18. In a preferred embodiment, the activating agent is a collagenase.

In a second aspect, the present invention provides a tissue scaffold or a tissue transplant device. Embodiments in accordance with this aspect of the invention generally include a biocompatible scaffold having a substrate cross-linked to a polypeptide growth factor by an enzymatic cross-linking agent. The growth factor is in an inactivated state in the scaffold.

Suitable polypeptide growth factors and enzymatic linking agents are as described above. Criteria for suitable substrate are the same as the peptide carrier described above.

In some embodiments, the scaffold may further incorporate cells. Exemplary cells that may be incorporated into the scaffold may include, but not limited to autologous cells mesenchymal or embryonic stems cells, progenitor cells, and primary cells. Preferably an undifferentiated progenitor cell.

Scaffolds of the present invention may be used simply as a storage or delivery vehicle for the growth factors or may serve as a transplant device in tissue repair applications.

In a third aspect, the present invention provides a system for storing a polypeptide growth factor. Embodiments in accordance with this aspect of the invention generally include a scaffold having a substrate capable of being covalently cross-linked to the polypeptide growth factor by an enzymatic cross-linking agent; and an enzymatic cross-linking agent for covalently cross-linking the polypeptide growth factor to the scaffold.

Polypeptide growth factors, enzymatic cross-linking agents, and substrates are as described above.

In a fourth aspect, the present invention provides a method for delivering a polypeptide growth factor in a controlled-release manner, Embodiments in accordance with this aspect of the invention generally include the steps of cross-linking the polypeptide growth factor to a substrate using an enzymatic cross-linking agent to form a storage or delivery vehicle loaded with said polypeptide growth factor; and introducing the storage or delivery vehicle to a target site for controlled-release of the polypeptide growth factor by an activating agent.

The target site may be either an in vivo, ex vivo, or in vitro. Physiological sites, cell culture, or any other environment suitable for cell growth and differentiation may all be selected as the target stie, Preferably, the site is a location of bone defect such as cleft palate, deformities, fractures, non-union defects, spinal fusion, bone fillers, cranial defects, or long bone segmental defects.

In some embodiments, a further step of incorporating cells into the storage or delivery vehicle may be taken. Suitable cells that may be incorporated into the storage or delivery vehicle are as described above. The polypeptide growth factors, substrates, enzymatic cross-linking agent, and activating agent are also as described above.

In a fifth aspect, the present invention provides a method for storing a polypeptide growth factor. Embodiments in accordance with this aspect of the invention generally include the steps of cross-linking the polypeptide growth factor to a substrate using an enzymatic cross-linking agent; and storing the cross-linked growth factor and peptide substrate in a lyophilized form.

The polypeptide growth factors, substrates, and enzymatic cross-linking agent are as described above. After cross-linking, the cross-linked peptides can be stored in any commonly known format of peptide storage. Exemplary format for storage may include, but not limited to solubilized solution with a stabilizing agent, crystal, or lyophilized powder. Preferably, the cross-linked peptides are stored in a lyophilized powder format for long-term stability, easy transportation, and storage.

In a sixth aspect, the present invention provides a method for fabricating a tissue transplant device. Embodiments in accordance with this aspect of the present invention generally include the steps of cross-linking a polypeptide growth factor to a scaffold having a substrate by using an enzymatic cross-linking agent.

In some embodiments, a further step of incorporating cells into the scaffold may be taken.

In still some further embodiments, a step of adding an activating agent to the scaffold may also be taken if differentiation and growth of cells on the scaffold is desired.

The polypeptide growth factors, substrates, cells, activating agent, and the enzymatic cross-linking agent are as described above.

In a seventh aspect, the present invention provides a method for tissue repairing or engineering. Embodiments in accordance with this aspect of the invention generally include the steps of placing a scaffold at a site in need of tissue repair or remodeling, wherein the scaffold is one that contains a polypeptide growth factor covalently linked to a substrate by an enzymatic cross-linking agent.

Sites in need of tissue repair or remodeling are generally in vivo sites, but may also be ex vivo sites. Exemplary sites may include wound locations or locations of tissue/organ defects. Preferably, the site in need of tissue repair is one with endogenously produced activating agent. To further control the repair process, an exogenously provided activating agent may also be added to the scaffold. The growth factors, substrates and sites and enzymatic cross-linking agents are as described above.

The compositions, devices, systems and methods of the present invention will have at least the advantages that release of the growth factors are controlled by the activating agents, thereby, avoiding the initial burst problem in conventional scaffolds or other delivery vehicles. Because

Other aspects and advantages of the invention will be apparent from the following description and the appended claims.

### BRIEF DESCRIPTION OF THE DRAWINGS

FIG. 1 shows de-activation of BMP-2 by gelatin and TGase. BMP-2 activity was determined by ALP activity from the C2C12 cell based BMP-2 activity assay. (A). BMP-2 alone, (B). gelatin mixed with BMP-2, (C). gelatin alone, (D). TGase alone, (E). TGase crosslinked gelatin, (F). BMP-2 with TGase and (G). gelatin-BMP-2 complex are presented. The error bars represent SD (n=5).

FIG. 2 shows re-activation of BMP-2 from the gelatin-BMP-2 complex by collagenase. (A). BMP-2 activity of BMP-2 control, (B). gelatin/BMP-2, (C). gelatin-BMP-2 complex, (D). gelatin-BMP-2 complex treated with collagenase, and (E). BMP-2 treated with collagenase are presented. Final concentration of collagenase was 1 U/ml when incubated with the gelatin-BMP-2 complex. Aliquots were taken from the incubated solution and added to C2C12 cell based BMP-2 activity assay. The error bars represent SD (n=5).

FIG. 3 shows dose response relationship of re-activated BMP-2 activity and collagenase concentration. The indicated concentrations were the final concentrations of collagenase that was incubated with gelatin-BMP-2 complex. Aliquots were retrieved after one hour of incubation at 37° C. for each collagenase concentration before subjected to C2C12 cell based BMP-2 activity assay. The Pearson correlation coefficient and associated p-value were r=0.8870 and p <0.001. Each point represented the mean ±SD (n=4).

FIG. 4 shows time course re-activation of BMP-2 from gelatin-BMP-2 complex by collagenase. The gelatin-BMP-2 complex was incubated at 37° C. with collagenase with a final incubation concentration of 1 U/ml for all time points. Aliquots were retrieved at various time points and subjected to C2C12 cell based BMP-2 activity assay The Pearson correlation coefficient and associated p-value were r=0.7388 and p <0.001. Each point represented the mean ±SD (n=4).

FIG. 5 shows cross-linking reaction between BMP-2 and gelatin by TGase and their collagenase digested products. SDS-PAGE gradient (3%-18%) was run at 90 V under non-reducing conditions. Lane 1: Molecular weight marker. Lane 2: BMP-2 alone. Lane 3: Gelatin alone. Lane 4: TGase alone. Lane 5: Collagenase alone. Lane 6: Gelatin/BMP-2. Lane 7: TGase crosslinked gelatin. Lane 8: Gelatin-BMP-2 complex. Lane 9: Gelatin-BMP-2 complex treated with collagenase. Lane 10: TGase crosslinked gelatin treated with collagenase. Lane 11: Gelatin treated with collagenase. Lane 12: BMP-2 treated with collagenase,

FIG. 6 shows re-activation of BMP-2 from gelatin-BMP-2 complex by tissue derived MMPs. A. Gelatin zymograph (10%) showed native MMPs secretion from rat skin organ culture for 1-5 days, in lane 1-5 respectively. B. Re-activated BMP-2 activity from gelatin-BMP-2 complex by tissue derived MMPs. The MMPs, which doses were represented in the zymograph, were incubated with the gelatin-BMP-2 complex for 1 hour before C2C12 cell based BMP-2 activity assay. The collected organ culture medium was tested for cell secreted BMP activity. “No MMP” refers to gelatin-BMP-2 complex in fresh medium. “BMP-2 with gelatin” served as positive control of BMP-2 without TGase crosslinking. The error bars represent SD (n=4).

FIG. 7 shows ectopic bone formation of gelatin-BMP-2 complex. TGase crosslinked gelatin sponge was used as a carrier for gelatin-BMP-2 complex. Explants underwent histology assay and ALP assay after 35 days. H&E staining of TGase crosslinked gelatin sponge (A) and gelatin-BMP-2 complex co-lyophilized with crosslinked gelatin (B). New bone formations are indicated with arrows. Scale bar=100 μm. ALP activity from the explants was shown in (C). All values represented in graphs are the mean ±SD (n=6).

FIG. 8 shows a schematic illustration of the de-activation and re-activation of BMP-2. Active signal molecules, such as BMP-2, are proportionally mixed with protective peptides, such as gelatin. After enzymatic crosslinking by TGase, the gelatin tethers to BMP-2 rendering it to become de-activated. The BMP-2 is re-activated by MMPs digestion secreted from the infiltrated cells. The consequential release of active BMP-2 regulates the cellular functions in the microenvironment.

FIG. 9 shows controlled reactivation of the polypeptide growth factors.

FIG. 10 shows BSA binding to BMP-2.

FIG. 11 shows the reactivation ability of different proteases.

FIG. 12 shows the carrier substrate as a protective layer.

FIG. 13 shows the release of MMP.

FIG. 14 shows cellular differentiation as a function of BMP-2 levels.

### DETAILED DESCRIPTION

General Methods and Utilities

The growth factors are generally cross-linked to the carrier substrate by simply adding a suitable TGase to a mixture of the growth factors and the carrier substrate in a suitable medium under appropriate reaction conditions.

For example, in an exemplary embodiment, microbial TGase was added to a blend of BSA and BMP2 to form BSA-BMP2 complexes. The reaction took place in a 1% solution of BSA, BMP2, PBS, and TGase in a ratio of 5:2:2:1 and was incubated overnight at room temperature. The BMP2 activity is de-activated in the resulting BMP2-BSA complex. This complex form an exemplary delivery system of this invention and may be introduced to a tissue repair or regeneration site. Once placed in the physiological environment, the complex will be broken down by enzymes in the native environment to release the BMP2. The released BMP2 regains its activity and performs its bone induction function.

Tissue regeneration scaffolds may be similarly prepared to embed de-activated growth factors prior to use in repair sites.

Prepackaged kits of growth factors and TGases may also be beneficially prepared for commercial distribution. Exemplary kits may include growth factor and TGases combinations suitable for a predetermined type of carrier substrate. These kits will have the advantage of providing convenience in both research and production settings.

The following specific experimental observations further illustrates this invention.

### EXAMPLES

BMP-2 Is De-Activated When Crosslinked to Gelatin by Transglutaminase

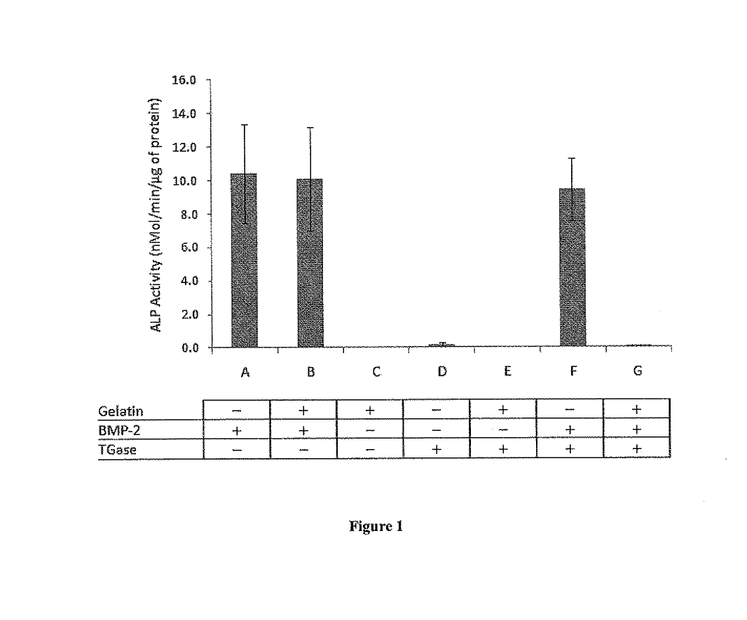

BMP-2 is known to transdifferentiate a premyoblast C2C12 cell line by dose dependently increasing alkaline phosphatase (ALP) activity. BMP-2 alone (FIG. 1A), gelatin/BMP-2 (FIG. 1B); and BMP-2 treated with TGase (FIG. 1F) all induced ALP activity at comparable levels. The high ALP activity suggested that TGase or gelatin had no inhibitory effect on BMP-2 activity. However, when BMP-2 and gelatin mixture was treated with TGase, BMP-2 activity was completely lost as exhibited by ALP (FIG. 1G). This indicated that as BMP-2 and gelatin bonded and formed a complex by the action of TGase, the formation of the complex (gelatin-BMP-2 complex) shielded the BMP-2 activity. Significant differences were observed between gelatin-BMP-2 complex and all other BMP-2 containing samples (p <0.001)

BMP-2 Is Re-Activated from a Gelatin-BMP-2 Complex by Bacterial Collagenase

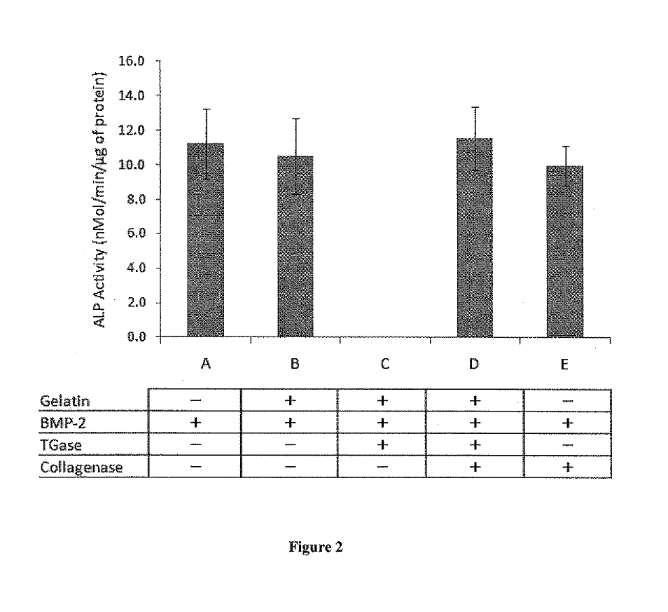

To determine whether BMP-2 activity can be restored, gelatin-BMP-2 complexes were digested by bacterial collagenase. As shown in FIG. 2, collagenase restored BMP-2 activity (FIG. 2D) to a level that did not significantly differ from free BMP-2 (FIG. 2A). Collagenase itself showed no effect on BMP-2 activity (FIG. 2E). This indicated that BMP-2 was not only de-activated by TGase-gelatin crosslinking to gelatin, but that it could also be re-activated by collagenase digestion.

Collagenase Dose- and Time-Dependent Re-Activation of BMP-2

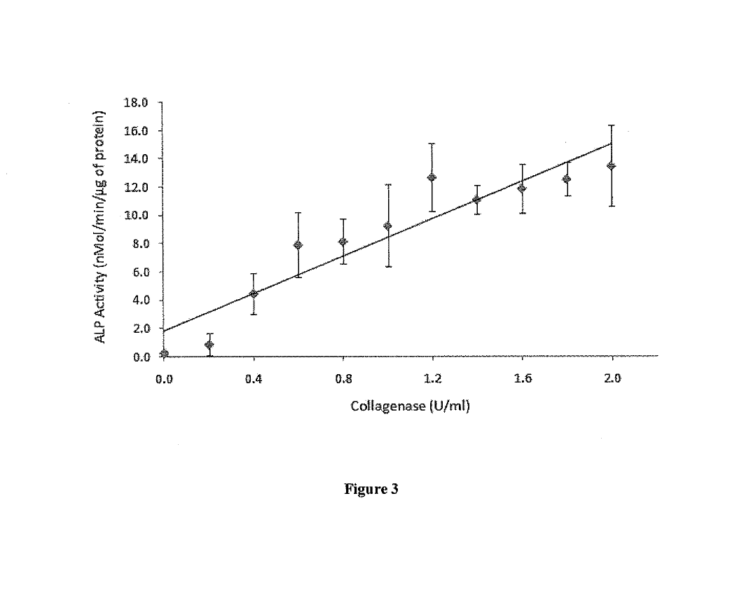

To demonstrate controllable activation, various amounts of collagenase were added to each gelatin-BMP-2 complex. FIG. 3 exhibits an increased BMP-2 activity as the dose of collagenase was increased (r=0.8870, p <0.001), showing that BMP-2 can be re-activated by collagenase in a dose dependent manner.

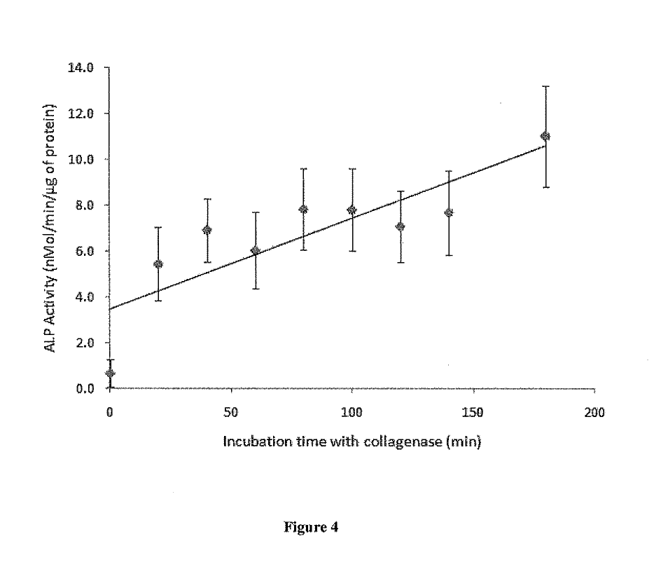

Temporal effects of re-activation of BMP-2 were evaluated by varying the incubation time using a defined concentration of collagenase. FIG. 4 shows BMP-2 activity to increase as incubation time was increased (r=0.7388, p <0.001), which demonstrates that a prolonged digestion results in increased BMP-2 re-activation from the gelatin-BMP-2 complex

SDS-PAGE Gel Examined the Gelatin-BMP-2 Complex Formation and Digestion

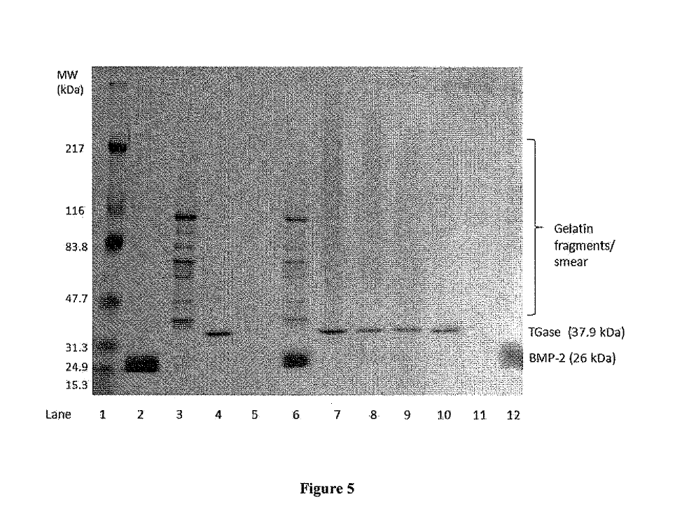

To elucidate the possible reaction mechanism, SDS-PAGE was used to monitor the protein complex formation and its digested byproducts after collagenase (FIG. 5). SDS-PAGE displayed BMP-2 with a molecular weight of 26 kDa in lane 2, gelatin as band fragments of different molecular weight peptides between 37 kDa and 116 kDa in lane 3 and TGase as a single band with molecular weight around 37 kDa in lane 4. Separate bands of BMP-2 and gelatin were viewed in lane 6 indicating that no binding transpired between BMP-2 and gelatin without TGase. The addition of TGase created a smear out of the fragmented gelatin peptide bands (lane 7) suggesting that TGase generated the crosslinking among the gelatin fragments. The same smear was observed in lane 8 (gelatin-BMP-2 complex), when BMP-2 and gelatin underwent the reaction by TGase.

Collagenase alone was run in lane 5 but concentration was not high enough to be visualized on the gel. However, the concentration was enough to digest the gelatin and TGase crosslinked gelatin as evidenced by the absence of bands in lane 10 and lane 11. The elimination of all bands of TGase crosslinked gelatin (lane 10) and gelatin (lane 11) show that collagenase completely digested gelatin and TGase crosslinked gelatin. However in lane 9 (gelatin-BMP-2 complex with collagenase), collagenase digestion left behind a smear indicating components of the complex to be indigestible. In comparing BMP-2 bands in lane 8 to lane 2 (BMP-2 alone) or lane 6 (gelatin/BMP-2), a fading band density of BMP-2 was observed. This suggested an uptake of BMP-2 as it binds to gelatin. Together with the effects associated with digestion, the smear on lane 9 most likely involved the bound BMP-2. Furthermore, BMP-2 in lane 12 showed no fragmentation or signs of digestion when treated with collagenase, suggesting BMP-2 was the un-degradable component left behind in lane 9. The smear also indicated that small undigested pieces of gelatin remained attached to BMP-2 after collagenase treatment.

MMPs re-activated BMP-2 from gelatin-BMP-2 complex

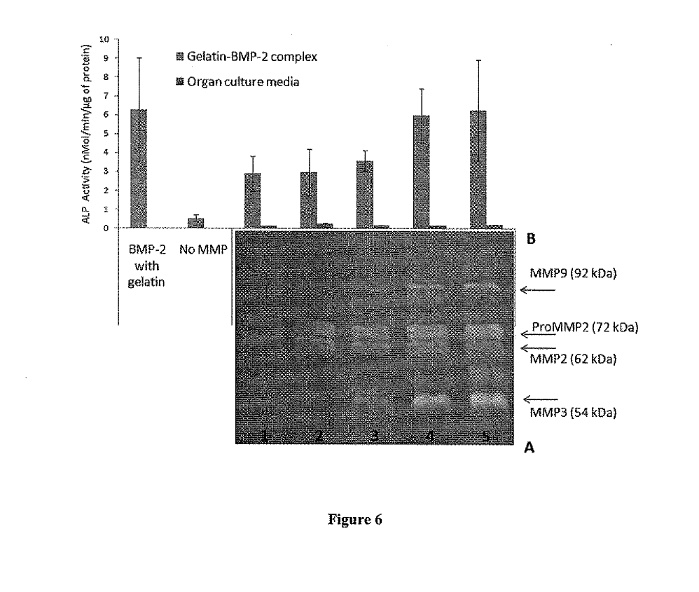

To test the effects of mammalian collagenase activity on gelatin-BMP-2 complex, MMPs were obtained from rat skin. Gelatin zymographs revealed that different types of MMPs were expressed from the dermis rat skin layer after prolonged incubation periods in organ culture. Signs of MMP2 production were observed from day 1. MMP9 and MMP3 began to manifest from day 2. All MMPs were observed to increase with longer organ culture incubation (FIG. 6A); thus a gradient of MMPs was created as daily collections from media were performed.

For the re-activation of BMP-2, the gelatin-BMP-2 complex was treated with a gradient of MMPs. Results show that native mammalian MMPs re-activated BMP-2 from the gelatin-BMP-2 complex in a dose dependent manner (FIG. 6B).

Ectopic Bone Formed with Gelatin-BMP-2 Complex

Before conducting the in vivo bone induction test for the gelatin-BMP-2 complex, samples of gelatin-BMP-2 complex before lyophilization were taken and tested by the C2C12 cell based BMP-2 activity assay. The results confirmed that BMP-2 was completely de-activated and that collagenase digestion of the samples led to re-activation (data not shown). Gelatin crosslinked by TGase was co-lyophilized with a pre-fowled gelatin-BMP-2 complex before implantation.

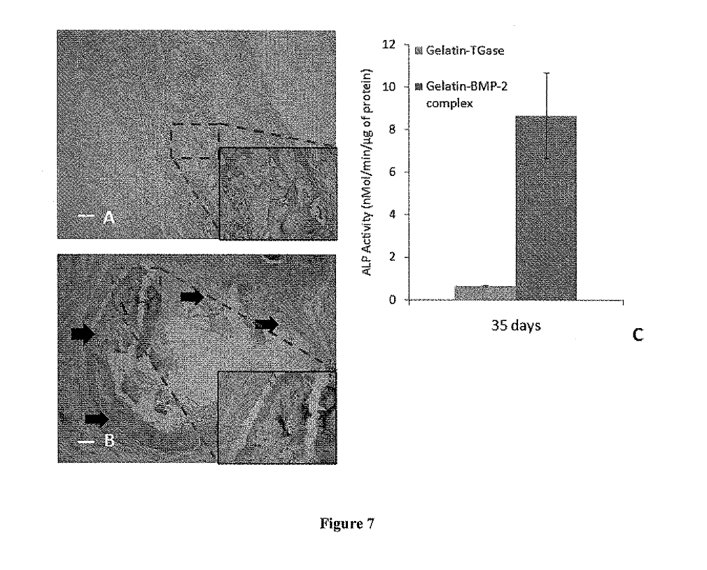

After 35 days of implantation in the abdominal muscle pouch, samples were explanted and stained. H&E staining showed active cellular infiltration around and inside all implants. For scaffold alone, we observed it to be partially degraded with fibrous tissue ingrowths and no bone formation (FIG. 7A). However, when implants were supplemented with gelatin-BMP-2 complex, new bone formation was clearly evident. Interestingly, areas of new bone formation were focused at the outer edge of the implant (FIG. 7B). The lack of bone formation in the center part of implant indicated that cell-instructive activation only occurred on the surface of the construct under the current design and time course of the study. The results of ALP activity from explants correlated with the histological findings. Gelatin-BMP-2 complex group exhibited higher levels of ALP activity compared to scaffold alone (FIG. 7C), indicating active bone formation in the gelatin-BMP-2 complex containing explants.

Trypsin Dose- and Time-Dependent Re-Activation of BMP-2

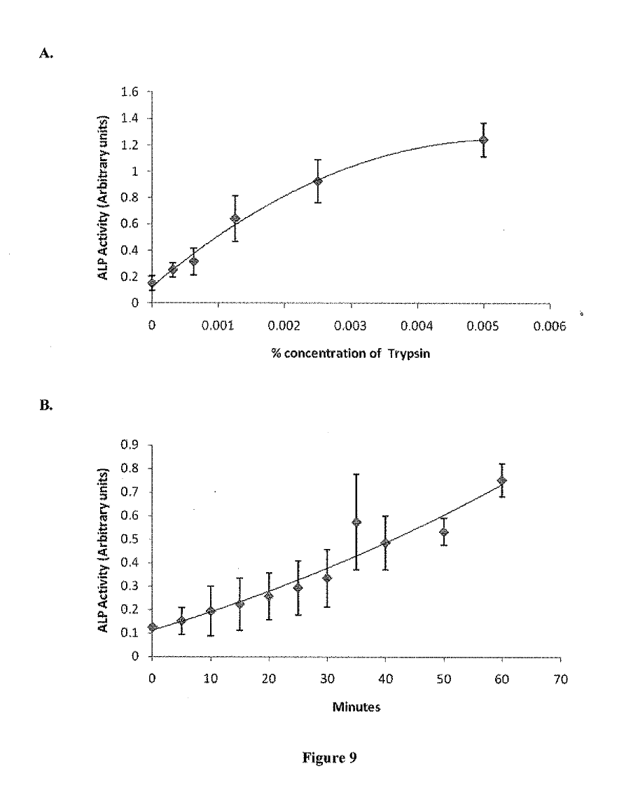

To demonstrate controllable re-activation with another enzyme other than collagenase, various amounts of trypsin were added to each gelatin-BMP-2 complex. FIG. 9 exhibits an increased BMP-2 activity as the dose of trypsin was increased, showing that BMP-2 can be re-activated by collagenase in a dose dependent manner.

Temporal effects of re-activation of BMP-2 were also evaluated by varying the incubation time using a defined concentration of trypsin. FIG. 9 shows BMP-2 activity to increase as incubation time was increased, which demonstrates that a prolonged digestion results in increased BMP-2 re-activation from the gelatin-BMP-2 complex.

BMP-2 Is De-Activated When Bound to BSA and Re-Activated With Trypsin

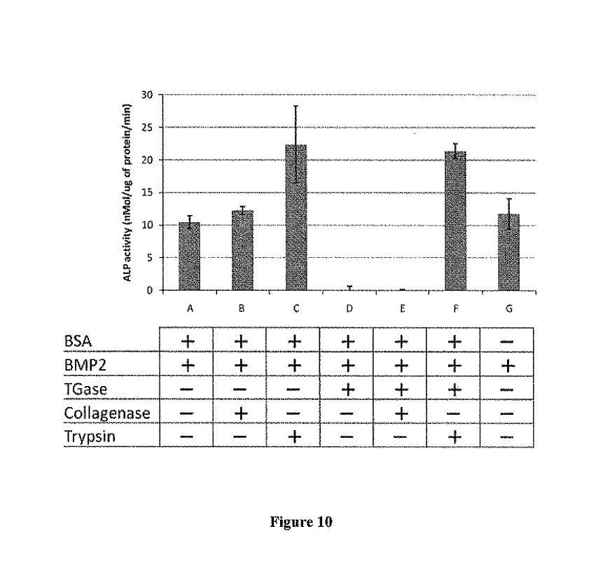

To confirm BSA's role in the de-activation of BMP-2, BSA-BMP-2 complexes were prepared and tested on the C2C12 ALP assay. Samples of BMP-2 alone and BSA mixed with BMP-2 were also taken as positive controls. For re-activation, one sample of the BSA-BMP-2 complex was incubated with 1 U/mL of collagenase and another with 0.025% concentration of trypsin for 1 hour at 37° C. In FIG. 10, the formation of BSA-BMP-2 complex resulted in the de-activation the BMP-2 activity. However, unlike the gelatin based complex, collagenase did not restore the BMP-2's activity. BMP-2 activity was re-activated after the adding the trypsin to the BSA-BMP-2 complex. Because BSA is digested by trypsin and not by collagenase, this results further indicates that the protein that binds to the BMP-2 can selectively re-activate BMP-2 depending on the proteases it reacts with.

BMP-2 Is De-Activated When Bound to BSA and Re-Activated With Trypsin

In the previous study, BMP-2 activity was re-activated by the addition of collagenase. In this study, other proteases were sought out and incubated with the gelatin-BMP2 and the BSA-BMP-2 complexes, BMP-2 activity was determined to observe if BMP-2 could be re-activated by proteases such as pronase, collagenase, trypsin, chymotrypsin, papain, and chymopapain.

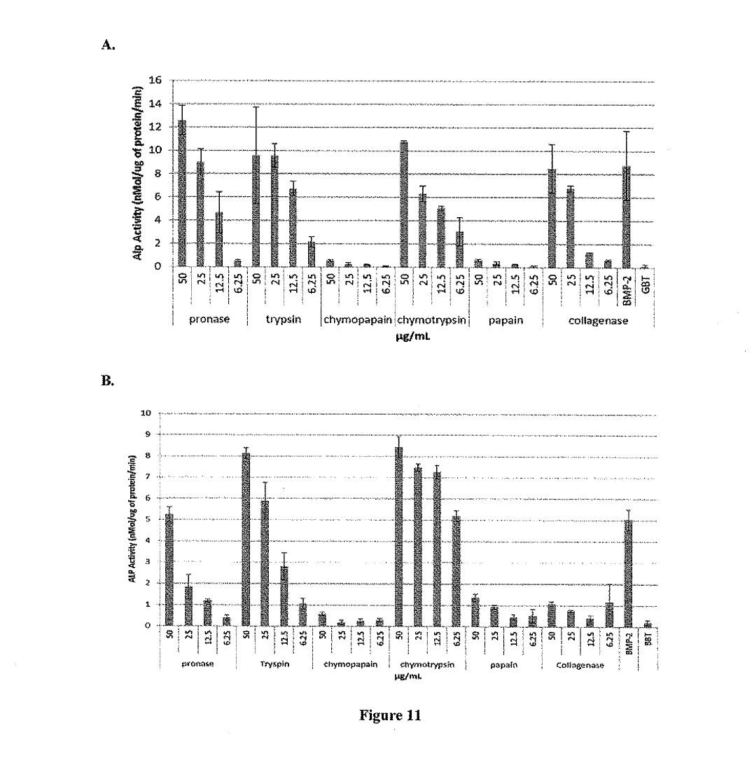

For gelatin-BMP2-TGase, higher concentration of trypsin and collagenase was shown to re-activate more BMP-2 activity for gelatin-BMP-2-TGase complex In FIG. 11, Pronase and chymotrypsin also showed a controllable re-activation of BMP-2 activity. Although the chymopapain and papain show low BMP-2 activity, later studies have indicated that the protease digests BMP-2. Lower dosages of chymopapain and papain may show protective properties of the complex.

For BSA-BMP-2 complex, results indicated that BMP-2 could also be controlled and reactivated by other proteases (FIG. 11). Similarily with gelatin-BMP-2 complex, chymopapain and papain did not re-activate BMP-2 from the BSA-BMP2 complex. Chymotrypsin, trypsin and pronase showed re-activity of BMP-2 and dosage control. Again, the result repeatedly showed that unlike gelatin-BMP2 complex, collagenase did not re-activate BMP-2 from BSA-BMP-2 complex.

Carrier Substrate as a Protective Layer

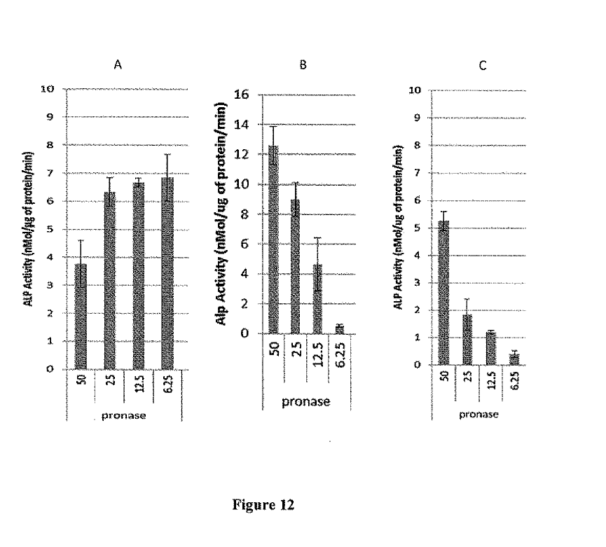

In observing pronase digestion on both gelatin and BSA complexes, BMP-2 activity was re-activated as the concentration of pronase were increased, (FIG. 11) However, when unbound BMP-2 was added to pronase, the BMP-2 activity decreased as the added pronase concentration increased (FIG. 12). This suggests BMP-2 is digested by pronase. Pronase is relatively non-selective in digestion of proteins and can digest many types of proteins including BMP-2, BSA and gelatin Taken together, the results suggest that the BSA or gelatin protein may have protected the BMP-2 by having the carrier being digested first. The carrier substrate not only have a role as switch but also act as a protectant of BMP-2.

Releases of MMP

In a previous study (Kuwahara, Yang et al., 2010), MSCs were successfully encapsulated by gelatin-TGase and delivered to repair sites. MSC in this study was replaced with C2C12 to better assess BMP-2 activity by ALP activity. Gelatin-BMP2 complex was made separately with 2% gelatin solution, producing a liquid-like consistency. Aliquots of gelatin-BMP-2 complex with BMP-2 at 4 ng/ul and 10 ng/ul concentration were tested and was shown to be de-activated. The gelatin-BMP2 complex was also subjected to collagenase and was shown to re-activate BMP-2. When C2C12 were encapsulated in a 10% gelatin crosslinked with TGase, the gelatin-BMP-2 complex was also included in the mix. The gelatin-TGase gel containing the gelatin-BMP-2 complex and C2C12 were incubated in wells.

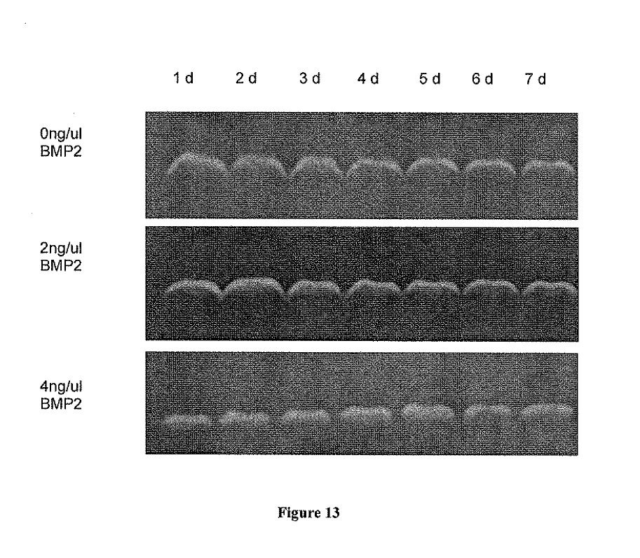

The surrounding media of the gelatin-TGase gels was taken daily for detecting digestive enzymes released by C2C12. The zymograph results show that MMP2 was released daily from C2C12 (FIG. 13), This suggests the MMP2 released by the C2C12 can re-activate the BMP-2 in the gelatin-BMP-2 complex.

Cellular Differentiation

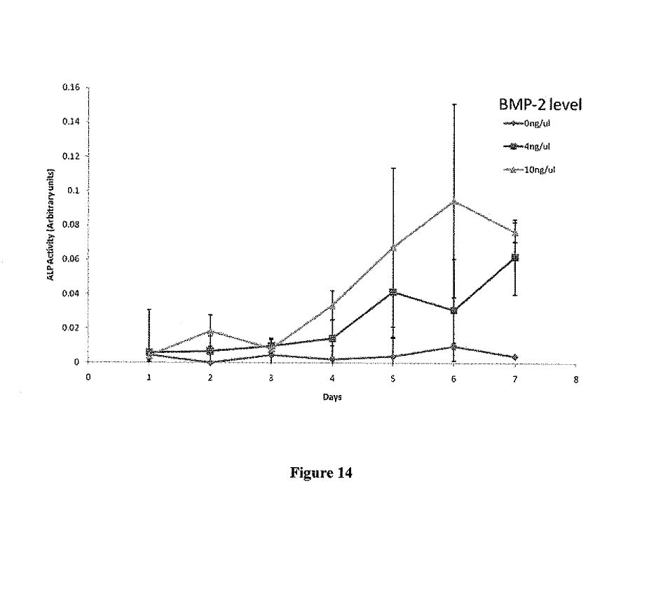

ALP activity from the harvested C2C12 showed that C2C12 was able to activate ALP activity from the BMP-2 that was bound in the gelatin-BMP-2 complex (FIG. 14). ALP activity started to increase after the third day for the C2C12 encapsulated containing 10 ng/ul BMP2 and the fourth day for the 4 ng/ul BMP2. The C2C12 with no gelatin-BMP2-TGase had no ALP activity. The ALP activity of the encapsulated C2C12 increased, as the encapsulation time for C2C12 became longer. Taken together with the MMP2 released by C2C12 (FIG. 13), C2C12 differentiated toward a osteoblastic lineage as the MMPs released by C2C12 were re-activating the BMP-2.

### Discussion

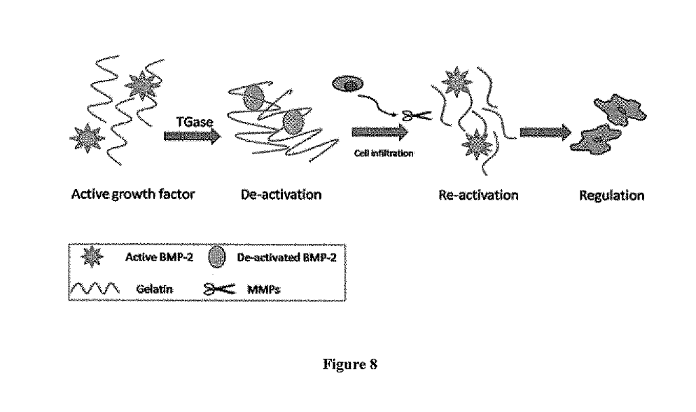

Immobilization of soluble regulatory peptides on carrier molecules or scaffolds which are later released by cellular activation may provide a powerful means to control cell behavior and enable complex processes of tissue formation and regeneration. The examples described herein is the first report of the novel discovery that the activity of growth factor when conjugated to a carrier protein by enzymatic action can be switched off converting from an active ligand into its latent form. Surprisingly, through the action of proteolytic action such as MMPs, the growth factor can be re-activated into its full potential. Schematic mechanism is illustrated in FIG. 8. This new drug delivery method by immobilization of bioactive ligand onto a carrier molecule not only changes its biomolecular characteristics (active-latent-active) but also creates signal controlling microenvironment by cell matrix interaction.

Embodiments of the present invention uses a native form of growth factor. In contrast, all previously known method of enzymatically regulated release of growth factors requires the engineering of new fusion proteins that contain exogenous substrates for enzymatic crosslinking and/or for release by enzymatic degradation. In addition to the extra protein expression work, utilizing structurally modified recombinant proteins may be of concern for clinical applications. Embodiments of the present invention clearly demonstrates that the shielding of growth factor activity from cells can be achieved through binding a soluble biomaterial to the growth factor at the molecular level. While not intending to be bound by any particular theory, we hypothesize that the active sites of the growth factors are masked with protective peptide, gelatin, upon cross-linking. This contrasts with the traditional methods of using solid or semi-solid biomaterials for creating a separation barrier between the growth factor and cells.

The TGase exhibits its effect by covalently binding the ε-amino group of a lysine residue to the γ-carboxamide group of a glutamine. However, for most TGase protein reactions, TGase was found to be highly selective towards only one single or rather few glutamines (Q) among the many other glutamine residues present in proteins. The substrate reactivity toward TGase is determined by the amino acids surrounding the glutamine in the peptide chain. Repeated glutamines were shown to increase reactivity with TGase. Ohtsuka et al. reported that the substrate reactivity is enhanced when the leucine (L), glutamic acid (E) or valine (V) is placed in front of the Q. Ito et al. enhanced microbial TGase crosslinking of RGD to gelatin by synthesizing a peptide with the Ls in front the Q as seen in the resulting sequence RGDLLQ. TGase also shows reactivity preference toward different lysines depending on their location or sequence. If growth factors have endogenous reactive sites for TGase to bind to, the growth factor can be tethered onto a suitable reactive scaffold like gelatin or collagen which contain both reactive lysine and glutamine residues. In embodiments of this invention, unmodified native BMP-2 was crosslinked directly onto gelatin, revealing that endogenous TGase reaction sites are available on BMP-2.

The de-activation of the growth factor as a result of this crosslinking reaction was initially discouraging, but the later re-activation of BMP-2 after the addition of collagenase brought to light an interesting phenomenon. Because collagenase was found to be selective towards digesting both TGase crosslinked and non-crosslinked gelatin and had no effect on BMP-2, gelatin appears to be responsible for de-activation. Although the binding sites where gelatin attaches onto BMP-2 still need to be elucidated, they do not seem to be binding to the active sites of BMP-2. Covalent bonds between ε-amino group and γ-carboxamide group are not digestible by collagenase, and if the bonds occurred at the BMP-2 active site, the residual gelatin fragment after digestion would have blocked the BMP-2 active site permanently. Biological active regions of BMP-2 are reported to be located on the two epitopes, the wrist epitope and knuckle epitope. The wrist epitope associates and binds with the BMP receptor IA and the knuckle epitope associates with the BMP receptor type II. It has been shown that mutating regions of one or the other epitope of BMP-2 reduces but not entirely demolishes the biological effect of BMP-2. In embodiments of this invention, BMP-2 activity in the gelatin-BMP-2 complex was completely lost. Since C2C12 cells have both BMP receptor type IA and II, it is possible that the bound gelatin, which serves as a latent sequence of BMP-2, shielded both BMP-2 epitopes that binds to the C2C12 cell receptors. Although BMP-2 activity assays and protein chromatography seems to support this pathway, the detailed mechanism requires further investigation.

Construction of MMP-sensitive biomaterial for guided tissue repair has been drawn a lot of attention in recent years. As a denatured form of collagen, gelatin is susceptible to degradation by various proteolytic enzymes, including MMPs. In our study, we demonstrated that, besides bacterial collagenase, gelatin-BMP-2 complex can be readily activated by tissue derived MMPs. MMPs are usually released by cells at the defect site for purposes of tissue repair and remodeling. Besides their role in remodeling, MMPs affect other cell functions such as proliferation and apoptosis. Secreted as inactive proenzymes and activated near the cell surface or expressed at the surface in activated form as membrane-type MMPs (MT-MMP), these enzymes can cleave virtually all constituents of the ECM.

In exemplary embodiments this invention, ECM derived collagen served dual roles, both as scaffold and growth factor switch. Our implant data clearly demonstrates that a TGase crosslinked gelatin scaffold can induce active cell infiltration and regeneration as well as support new bone formation. Overall, the multistep osteoinductive events can be described as follows: BMP-2 is covalently immobilized and the bioactive osteoinductive signal switched off in the gelatin matrix when implanted. The MMP-sensitive TGase crosslinked gelatin scaffold guides the inflammatory and osteoprogenitor cells to migrate into the matrix. The cells produced the MMPs which in addition to degrading the matrix released BMP-2 into its active form. BMP-2's osteoinductive signaling causes the differentiation of the osteoprogenitor cells in this microenvironment into osteoblasts.

METHODS

Preparation of Materials

Gelatin (Type B 225 bloom, Sigma Aldrich) was dissolved and autoclaved in distilled water to make a 10% gelatin stock. The autoclaved gelatin was aliquoted and stored at 4° C. until use. A 2% percent gelatin solution was made by diluting from a 10% gelatin stock at 37° C. with BMP-2 buffer (25 mM tricine, pH 7.2, 15 mM sucrose, 1.7 mM NaCl, and 0.01% Tween 80).

Microbial transglutaminase (ACTIVA TI Ajinomoto, Japan, TGase) from Streptomyces mobaraense was purified using a Sepharose Fast Flow column [47]. Briefly, 3 g of crude TGase were dissolved in a phosphate buffer (20 mM phosphate and 2 mM EDTA, pH 6.0) and gently mixed with 3 ml of pre-equilibrated S Sepharose Fast Flow beads (Sigma). After incubation at 4° C. overnight with occasional vortexing, the protein solution and beads mixture were batch loaded into a column. After washing with 4 volumes of phosphate buffer, TGase was eluted with eluting buffer (phosphate buffer with 800 mM NaCl). Protein concentration was monitored by the Bradford method (Bio-Rad) utilizing BSA as a standard. BMP-2 (R&D systems) was kept in stock concentrations of 20 ng/μl at −20° C. in buffer solution (5 mM glutamic acid, 2.5% glycine, 0.5% sucrose, and 0.01% Tween 80).

Preparation of Gelatin/BMP-2 and Gelatin-BMP-2 Complex

Gelatin/BMP-2: Two percent gelatin was mixed with 20 ng/μl BMP-2 stock at a ratio of 5:2 at room temperature. The final BMP-2 concentration was 4 ng/μl.

Gelatin-BMP-2 complex: The covalent binding of BMP-2 to gelatin was prepared using TGase. TGase was added to the gelatin/BMP-2 mixture at a final concentration of 25 μg/ml. The reaction was carried out at room temperature for 18 hours. Mixtures were either prepared fresh or stored at −80° C. before determining BMP-2 activity by using the C2C12 cell assay.

Dosage and Time Response Using Collagenase

Gelatin-BMP-2 complex was treated with collagenase to evaluate BMP-2 release and its activation profile. The complex was treated with 1 U/ml (final) bacterial collagenase (190 U/mg, Type 2, Worthington) at 37° C. for 1 hour. Samples were collected and taken for the C2C12 cell based BMP-2 activity assay.

For the dose response study, the gelatin-BMP-2 complex was treated with collagenase (0.2-2 U/ml for 1 hour at 37° C.) before samples were taken to the C2C12 cell based BMP-2 activity assay. For the time course study, a final concentration of 1 U/ml collagenase was added to separate aliquots of the gelatin-BMP-2 complex solution. The solution was incubated at 37° C. and retrieved at various time points from 0 to 180 minutes, and stored at −80° C. before assaying for BMP-2 activity.

SDS-PAGE Chromatography

To examine the BMP-2 interaction with gelatin, sample concentrations were increased because concentrations used in the C2C12 cell based BMP-2 activity assay were too low to be visible on SDS-PAGE. The final concentrations of BMP-2 and TGase in the mixtures were raised to 100 ng/μl and 40 μg/ml respectively. Gelatin (1 mg/ml) was generated from type I from rat tail collagen (lab prepared) by heat denaturation at 55° C. for 4 hours. Collagenase final concentration was 1.5 U/ml.

Reactants were mixed at 1:1 (v/v) with SDS sample buffer (125 mM Tris pH 6.8, 2% SDS, 0.1% bromophenol blue, and 25% glycerol) and heated at 100° C. for 5 min prior to loading onto the 3-18% gradient SDS-PAGE gel. The samples underwent electrophoresis at 90 V until the frontier reached the end of the gel. The gels were stained with Coomassie Blue solution (62.5% methanol, 25% acetic acid and 0.125% Coomassie Blue R250 (Bio-Rad)) overnight and destained with 30% methanol and 1% formic acid for 5 hours.

Extraction of Rat Skin Matrix Metalloproteinase (MMPs)

Within two hour after the euthanization of a Fisher 344 rat, a 2 cm×2 cm square piece of skin was removed from the abdominal area after being shaved and the surface-treated with 70% ethanol. The dermis was separated from the keratin layer and the attached muscle with a scalpel. The dermis was incubated in PBS containing a 2% antibiotic-antimycotic solution (Mediatech) at 4° C. for 15 hours. The skin was subsequently rinsed twice with PBS, excised into 0.5 cm×0.5 cm pieces and placed into a 60 mm tissue culture plate with 5 ml of DMEM containing 10 ng/ml (final) of TNFα (R&D Systems). Aliquots of 200 μl were collected after 1, 2, 3, 4 and 5 days of incubation at 37° C. 5% CO₂. MMPs were measured by a gelatinolytic zymograph.

Gelatin-BMP-2 Complex Treatment With Tissue Derived MMPs

Samples of 20 μl from the collected organ culture media were added to each of 100 μl aliquots of gelatin-BMP-2 complex and incubated at 37° C. for 1 hour. A subsequent C2C12 cell based BMP-2 activity assay was conducted on the gelatin-BMP-2 complex solution treated with organ culture media. Collected organ culture media without gelatin-BMP-2 complex was also assayed for BMP-2 activity as a control.

Gelatinolytic Zymograph Assay

Media collected from the organ culture were analyzed for MMP activity through a gelatin zymograph as described [48]. In short, the culture media was mixed with sample buffer at a 1:1 ratio (250 mM Tris pH 6.8, 10% SDS, 50% glycerol, 2.5 mg/ml of bromophenol blue) without reducing agent or heating. The sample was loaded into a gelatin (5.50□ mg/ml) containing 10% acrylamide/biascrylamide (Bio-Rad) and underwent electrophoresis with constant voltage (90 V) for 5.5 hours. Afterwards, the gel was washed with 2.5% Triton X-100 to remove the SDS, rinsed with 500 mM Tris-HCl pH 7.5, and then incubated overnight at 37° C. with the developing buffer (50 mM Tris pH 7.5, 5 mM CaCl₂, and 200 mM NaCl) for 16 hours. The zymographic activities were revealed by 1 hour staining with Coomassie Blue staining solution and subsequent overnight destaining with 30% methanol and 1% formic acid.

C2C12 Cell Based BMP-2 Activity Assay

BMP-2 dose dependently induces alkaline phosphatase (ALP) activity in a C2C12 mouse myoblast cell line [5]; therefore, activities of sequestered and released BMP-2 can be determined by ALP assay with C2C12 cells. C2C12 cells (ATCC) were seeded onto a 96-well-plate at a concentration of 1.25×10⁴ cells per well with 100 μl of 10% FBS/DMEM. The plate was incubated at 37° C., 5% CO₂ overnight for attachment. The media was exchanged to test media with 200 μl of 1% FBS/DMEM. Aliquots of 10 μl samples were added to each well and incubated for 48 hours. At the end of the incubation period, the media was removed, and the cells were washed twice with cold PBS. The cells were then lysed by adding 30 μl of 0.5% Triton X-100/PBS and undergoing three freeze/thaw cycles. For pNPP substrate solution, each pNPP tablet (5 mg p-nitrophenyl phosphate disodium salt/tablet, Thermo Scientific) was dissolved in 5 ml diethanolamine buffer (1.02 M diethanolamine 0.5 mM MgCl₂ pH 9.8). One hundred pi of pNPP substrate solution were added to the cell lysates and incubated at 37° C. for 30 min. ALP activity was determined by recording absorbance at 405 nm and normalized by protein content using a RCA Protein Assay kit (Bio-Rad).

Bone Induction of Gelatin-BMP-2 Complex In Vivo

Each gelatin-BMP-2 complex sample destined to be implanted was prepared by mixing 3 μg of BMP-2 (30 μl) into a 200 μl of 5% gelatin solution and incubated with TGase (25 μg/ml, final concentration) overnight. Before lyophilization, aliquots of the mixtures, with or without collagenase digestion, were assayed in vitro with C2C12 cells for BMP-2 activity to confirm BMP-2 binding and release. Lyophilized 5% gelatin gels that were crosslinked with 25 μg/ml of TGase were used as controls.

Animal use protocols were approved by IACUC of the University of Southern California. A total of 6 Fisher 344 rats (male, 8 weeks, wt 190-210 g) were used in the in vivo study. Muscle pouches were created in the abdominal muscles bilaterally at 6 sites by sharp and blunt dissection and subsequently packed with lyophilized gels. Samples were harvested after 35 days. Each sample was divided into two, one half fixed in 10% neutral buffered formalin for histology (H&E) and the other half homogenized in 0.5% Triton X-100 for ALP activity using p-nitrophenyl-phosphate (pNPP) as a substrate. Absorbance was measured at 405 nm after 15 min of incubation at 37° C. The activity was normalized by total protein content using a BCA Protein Assay kit (Bio-Rad).

Statistical Analysis

The student t-test was performed to evaluate differences in all C2C12 cell based BMP-2 activity assays. Data were expressed as mean ±SD. The Pearson correlation test was used for evaluating the time and dose dependent effects of collagenase digestion. Statistical significance was set at p <0.05 in all analyses.

Although the present invention has been described in terms of specific exemplary embodiments and examples, it will be appreciated that the embodiments disclosed herein are for illustrative purposes only and various modifications and alterations might be made by those skilled in the art without departing from the spirit and scope of the invention as set forth in the following claims.

### REFERENCES

The following references are incorporated herein by reference.

1. Kugimiya, F., et al., Involvement of endogenous bone morphogenetic protein (BMP) 2 and BMP6 in bone formation. J Biol Chem, 2005. 280(42): p. 35704-12.

2. Urist, M. R., Bone: formation by autoinduction. Science, 1965, 150(698): p. 893-9,

3. Wang, E. A., et al., Bone morphogenetic protein-2 causes commitment and differentiation in C3H10T1/2 and 3T3 cells. Growth Factors, 1993. 9(1): p. 57-71.

4. Suzawa, M., et al., Extracellular matrix-associated bone morphogenetic proteins are essential for differentiation of murine osteoblastic cells in vitro. Endocrinology, 1999. 140(5): p. 2125-33.

5. Katagiri, T., et al., Bone morphogenetic protein-2 converts the differentiation pathway of C2C12 myoblasts into the osteoblast lineage. J Cell Biol, 1994. 127(6 Pt 1): p. 1755-66.

6. Yamaguchi, A., et al., Recombinant human bone morphogenetic protein-2 stimulates osteoblastic maturation and inhibits myogenic differentiation in vitro. J Cell Biol, 1991. 113(3): p. 681-7.

7. Otsuru, S., et al., Circulating bone marrow-derived osteoblast progenitor cells are recruited to the bone forming site by the CXCR4/stromal cell-derived factor-1 pathway. Stem Cells, 2008. 26(1): p. 223-34.

8. Sampath, T. K., et al., Recombinant human osteogenic protein-1 (hOP-1) induces new bone formation in vivo with a specific activity comparable with natural bovine osteogenic protein and stimulates osteoblast proliferation and differentiation in vitro. J Biol Chem, 1992. 267(28): p. 20352-62.

9. Wang, E. A., et al., Recombinant human bone morphogenetic protein induces bone formation. Proc Natl Acad Sci USA, 1990. 87(6): p. 2220-4.

10. Burkus, J. K., et al., Use of rhBMP-2 in combination with structural cortical allografts: clinical and radiographic outcomes in anterior lumbar spinal surgery. J Bone Joint Surg Am, 2005. 87(6): p. 1205-12.

11. Vaccaro, A. R., et al., Comparison of OP-1 Putty (rhBMP-7) to iliac crest autograft for posterolateral lumbar arthrodesis: a minimum 2-year follow-up pilot study. Spine (Phila Pa. 1976), 2005. 30(24): p. 2709-16.

12. Carlisle, E. and J. S. Fischgrund, Bone morphogenetic proteins for spinal fusion. Spine J, 2005. 5(6 Suppl): p. 2408-2495.

13. Braddock, M., et al., Born Again Bone: Tissue Engineering for Bone Repair. News Physiol Sci, 2001. 16(5): p. 208-213.

14. Yamamoto, M., Y. Takahashi, and Y. Tabata, Controlled release by biodegradable hydrogels enhances the ectopic bone formation of bone morphogenetic protein. Biomaterials, 2003. 24(24): p. 4375-83.

15. Yamamoto, M., Y. Tabata, and Y. Ikada, Ectopic bone formation induced by biodegradable hydrogels incorporating bone morphogenetic protein. J Biomater Sci Polym Ed, 1998, 9(5): p. 439-58.

16. Yang, S. H., et al., Tricalcium phosphate and glutaraldehyde crosslinked gelatin incorporating bone morphogenetic protein--a viable scaffold for bone tissue engineering. J Biomed Mater Res B Appl Biomater, 2005. 74(1): p. 468-75.

17. Seeherman, H. and J. M. Wozney, Delivery of bone morphogenetic proteins for orthopedic tissue regeneration. Cytokine Growth Factor Rev, 2005. 16(3): p. 329-45.

18. Kempen, D. H., et al., Retention of in vitro and in vivo BMP-2 bioactivities in sustained delivery vehicles for bone tissue engineering. Biomaterials, 2008. 29(22): p. 3245-52.

19. Kim, H. D. and R. F. Valentini, Retention and activity of BMP-2 in hyaluronic acid-based scaffolds in vitro. J Biomed Mater Res, 2002. 59(3): p. 573-84.

20. Nie, H., et al., Three-dimensional fibrous PLGA/HAp composite scaffold for BMP-2 delivery. Biotechnol Bioeng, 2008. 99(1): p. 223-34.

21. Rengachary, S. S., Bone morphogenetic proteins: basic concepts. Neurosurg Focus, 2002. 13(6): p. e2.

22. Luginbuehl, V., et al., Localized delivery of growth factors for bone repair. Eur J Pharm Biopharm, 2004. 58(2): p. 197-208.

23. Rosen, V., BMP and BMP inhibitors in bone. Ann N Y Acad Sci, 2006. 1068: p. 19-25.

24. Toth, J. M., et al., Short-term osteoclastic activity induced by locally high concentrations of recombinant human bone morphogenetic protein-2 in a cancellous bone environment. Spine (Phila Pa. 1976), 2009. 34(6): p. 539-50.

25. Liu, Y., et al., The influence of BMP-2 and its mode of delivery on the osteoconductivity of implant surfaces during the early phase of osseointegration, Biomaterials, 2007. 28(16): p. 2677-86.

26. Tuan, T. L., et al., Engineering, expression and renaturation of targeted TGF-beta fusion proteins. Connect Tissue Res, 1996. 34(1): p. 1-9.

27. Han, B., F. L. Hall, and M. E. Nimni, Refolding of a recombinant collagen-targeted TGF-beta2 fusion protein expressed in Escherichia coli. Protein Expr Purif, 1997. 11(2): p. 169-78.

28. Han, B., et al., Collagen-targeted BMP3 fusion proteins arrayed on collagen matrices or porous ceramics impregnated with Type I collagen enhance osteogenesis in a rat cranial defect model. J Orthop Res, 2002. 20(4): p. 747-55.

29. Hall, F.L., et al., Phenotypic differentiation of TGF-beta1-responsive pluripotent premesenchymal prehematopoietic progenitor (P4 stem) cells from murine bone marrow. J Hematother Stem Cell Res, 2001. 10(2): p. 261-71.

30. Andrades, J. A., et al., A modified rhTGF-beta1 and rhBMP-2 are effective in initiating a chondro-osseous differentiation pathway in bone marrow cells cultured in vitro. Connect Tissue Res, 2003. 44(3-4): p. 188-97.

31. Liu, H. W., et al., Targeted delivery system for juxtacrine signaling growth factor based on rhBMP-2-mediated carrier-protein conjugation. Bone, 2006. 39(4): p. 825-36.

32. Schmoekel, H. G., et al., Bone repair with a form of BMP-2 engineered for incorporation into fibrin cell ingrowth matrices. Biotechnol Bioeng, 2005. 89(3): p. 253-62.

33. Tabata, Y., Tissue regeneration based on tissue engineering technology. Congenit Anom (Kyoto), 2004. 44(3): p. 111-24.

34. Tabata, Y., S. Hijikata, and Y. Ikada, Enhanced vascularization and tissue granulation by basic fibroblast growth factor impregnated in gelatin hydrogels. Journal of Controlled Release, 1994. 31(2): p. 189-199.

35. Ferreira, J. P., et al., A study on the functional subunits of phospholipases A2 by enzyme immobilization. Biochem J, 1994. 303 (Pt 2): p. 527-30.

36. Tsujigiwa, H., et al., Effects of immobilized recombinant human bone morphogenetic protein-2/succinylated type I atelocollagen on cellular activity of ST2 cells. J Biomed Mater Res A, 2005. 75(1): p. 210-5.

37. Ehrbar, M., et al., Enzymatic formation of modular cell-instructive fibrin analogs for tissue engineering. Biomaterials, 2007. 28(26): p. 3856-66.

38. Demolliens, A., et al., Tyrosinase-catalyzed synthesis of a universal coil-chitosan bioconjugate for protein immobilization. Bioconjug Chem, 2008. 19(9): p. 1849-54.

39. Scuderi, N., et al., The clinical application of autologous bioengineered skin based on a hyaluronic acid scaffold. Biomaterials, 2008. 29(11): p. 1620-9.

40. Ehrbar, M., et al., The role of actively released fibrin-conjugated VEGF for VEGF receptor 2 gene activation and the enhancement of angiogenesis. Biomaterials, 2008. 29(11): p. 1720-9.

41. Sakiyama-Elbert, S. E., A. Panitch, and J. A. Hubbell, Development of growth factor fusion proteins for cell-triggered drug delivery. FASEB J, 2001. 15(7): p. 1300-2.

42. Kuwahara, K., et al., Cell delivery using an injectable and adhesive transglutaminase- gelatin gel. Tissue Eng Part C Methods, 2010. 16(4): p. 609-18.

43. Bertoni, F., et al., Transglutaminase Reactivity with Gelatine: Perspective Applications in Tissue Engineering. Biotechnol Lett, 2006.

44. Ito, A., et al., Transglutaminase-mediated gelatin matrices incorporating cell adhesion factors as a biomaterial for tissue engineering. J Biosci Bioeng, 2003. 95(2): p. 196-9.

45. Yung, C. W., et al., Transglutaminase crosslinked gelatin as a tissue engineering scaffold. Biomed Mater Res A, 2007. 83(4): p. 1039-46.

46. Chen, R. N., H. O. Ho, and M. T. Sheu, Characterization of collagen matrices crosslinked using microbial transglutaminase. Biomaterials, 2005. 26(20): p. 4229-35.

47. Chau, D. Y., et al., The cellular response to transglutaminase-cross-linked collagen. Biomaterials, 2005. 26(33): p. 6518-29.

48. Han, Y. P., et al., TNF-alpha stimulates activation of pro-MMP2 in human skin through NF-(kappa)B mediated induction of MT1-MMP. J Cell Sci, 2001. 114(Pt 1): p. 131-139.

49. Han, B., B. Tang, and M. E. Nimni, Quantitative and sensitive in vitro assay for osteoinductive activity of demineralized bone matrix. J Orthop Res, 2003. 21(4): p. 648-54.

50. Gobin, A. S. and J. L. West, Effects of epidermal growth factor on fibroblast migration through biomimetic hydrogels. Biotechnol Prog, 2003. 19(6): p. 1781-5.

51. Folk, J. E., Mechanism and basis for specificity of transglutaminase-catalyzed epsilon-(gamma-glutamyl) lysine bond formation. Adv Enzymol Relat Areas Mol Biol, 1983. 54: p. 1-56.

52. Fontana, A., et al., Site-specific modification and PEGylation of pharmaceutical proteins mediated by transglutaminase. Adv Drug Deliv Rev, 2008. 60(1): p. 13-28.

53. Kahlem, P., et al., Peptides containing glutamine repeats as substrates for transglutaminase-catalyzed cross-linking: relevance to diseases of the nervous system. Proc Natl Mad Sci USA, 1996. 93(25): p. 14580-5.

54. Ohtsuka, T., et al., Comparison of substrate specificities of transglutaminases using synthetic peptides as acyl donors. Biosci Biotechnol Biochem, 2000. 64(12): p. 2608-13.

55. Taguchi, S., et al., Substrate specificity analysis of microbial transglutaminase using proteinaceous protease inhibitors as natural model substrates. J Biochem, 2000. 128(3): p. 415-25.

56. Nickel, J., et al., The crystal structure of the BMP-2: BMPR-1A complex and the generation of BMP-2 antagonists. J Bone Joint Surg Am, 2001. 83-A Suppl 1(Pt 1): p. S7-14.

57. Namiki, M., et al., A kinase domain-truncated type I receptor blocks bone morphogenetic protein-2-induced signal transduction in C2C12 myoblasts. J Biol Chem, 1997. 272(35): p. 22046-52.

58. Kirsch, T., J. Nickel, and W. Sebald, BMP-2 antagonists emerge from alterations in the low-affinity binding epitope for receptor BMPR-II EMBO J, 2000. 19(13): p. 3314-24.

59. Lutolf, M. P., et al., Synthetic matrix metalloproteinase-sensitive hydrogels for the conduction of tissue regeneration: engineering cell-invasion characteristics. Proc Natl Acad Sci USA, 2003. 100(9): p. 5413-8.

60. Lutolf, M. P., et al., Repair of bone defects using synthetic mimetics of collagenous extracellular matrices. Nat Biotechnol, 2003. 21(5): p. 513-8.

61. Seliktar, D., et al., MMP-2 sensitive, VEGF-bearing bioactive hydrogels for promotion of vascular healing. J Biomed Mater Res A, 2004. 68(4): p. 704-16.

62. Kraehenbuehl, T. P., et al., Cell-responsive hydrogel for encapsulation of vascular cells. Biomaterials, 2009. 30(26): p. 4318-24.

63. Raeber, G. P., M. P. Lutolf, and T. A. Hubbell, Mechanisms of 3-D migration and matrix remodeling offibroblasts within artificial ECMs. Acta Biomater, 2007. 3(5): p. 615-29.

64. Zisch, A. H., et al., Cell-demanded release of VEGF from synthetic, biointeractive cell ingrowth matrices for vascularized tissue growth. FASEB J, 2003. 17(15): p. 2260-2.

65. Lluri, G. and D. M. Jaworski, Regulation of TIMP-2, MT1-MMP, and MMP-2 expression during C2C12 differentiation. Muscle Nerve, 2005. 32(4): p. 492-9.

66. Blumenfeld, I., et al., Metalloproteinases (MMPs-2, -3) are involved in TGF-beta and IGF-1-induced bone defect healing in 20-month-old female rats. Arch Gerontol Geriatr, 2002. 35(1): p. 59-69.

67. Kinoh, H., et al., MT-MMP, the cell surface activator of proMMP-2 (pro-gelatinase A), is expressed with its substrate in mouse tissue during embryogenesis. J Cell Sci, 1996. 109 (Pt 5): p. 953-9.

68. Murphy, G. and M. H. Lee, What are the roles of metalloproteinases in cartilage and bone damage? Ann Rheum Dis, 2005. 64 Suppl 4: p. iv44-7.

What is claimed is:

1. A composition for delivery and controlled-release of a polypeptide growth factor, comprising:
a therapeutically effective amount of a polypeptide growth factor; and
a biocompatible carrier substrate;
wherein said polypeptide growth factor is covalently cross-linked to the carrier substrate by a transglutaminase.

2. The composition of claim 1, wherein said polypeptide growth factor is one selected from the group consisting of BMP10, BMP15, BMP2, BMP3, BMP4, BMP5, BMP6, BMP7, BMP8A, BMP8B, TGFB1, TGFB2, TGFB3, GDF1, GDF10, GDF11, GDF15, GDF2, GDF3, GDF3A, GDF5, GDF6, GDF7, GDF8, GDF9, GDNF, INHA, INHBA, INHBB, INHBC, INHBE, LEFTY1, LEFTY2, MSTN, NODAL, NRTN, PSPN, AMH, ARTN, FOP, PDGF, VEGF, IGF, and NGF.

3. The composition of claim 1, wherein said polypeptide growth factor is a bone morphogenetic protein.

4. The composition of claim 1, wherein said polypeptide growth factor is BMP-2.

5. The composition of claim 1, wherein said polypeptide growth factor has an unmodified native polypeptide sequence.

6. The composition of claim 1, wherein said carrier substrate comprises a peptide with an exposed lysine or glutamine.

7. The composition of claim 1, wherein said carrier substrate is selected from collagen, gelatin, albumin, fibrin, fibrinogen, laminin, fibronectin, vitronectin or a synthetic peptide containing an exposed lysine or glutamine.

8. The composition of claim 1, wherein said transglutaminase is a bacterial transglutaminase.

9. A tissue scaffold or a tissue transplant device, comprising:
a biocompatible scaffold comprising a carrier substrate covalently cross-linked to a polypeptide growth factor by a transglutaminase,
wherein said polypeptide growth factor is inactivated upon being cross-linked to said substrate.

10. The tissue scaffold or transplant device of claim 9, wherein said polypeptide growth factor is one selected from the group consisting of BMP10, BMP15, BMP2, BMP3, BMP4, BMP5, BMP6, BMP7, BMP8A, BMP8B, TGFB1, TGFB2, TGFB3, GDF1, GDF10, GDF11, GDF15, GDF2, GDF3, GDF3A, GDF5, GDF6, GDF7, GDF8, GDF9, GDNF, INHA, INHBA, INHBB, INHBC, INHBE, LEFTY1, LEFTY2, MSTN, NODAL, NRTN, PSPN, AMH, ARTN, FGF, PDGF, VEGF, IGF, and NGF.

11. The tissue scaffold or transplant device of claim 9, wherein said polypeptide growth factor is a bone morphogenetic protein.

12. The tissue scaffold or transplant device of claim 9, wherein said polypeptide growth factor is BMP-2.

13. The tissue scaffold or transplant device of claim 9, wherein said carrier substrate comprises a peptide with an exposed lysine or glutamine.

14. The tissue scaffold or transplant device of claim 9, wherein said carrier substrate is selected from collagen, gelatin, albumin, fibrin, fibrinogen, laminin, fibronectin, vitronectin or a synthetic peptide containing an exposed lysine or glutamine.

15. The tissue scaffold or transplant device of claim 9, wherein said transglutaminase is a bacterial transglutaminase.

16. The tissue scaffold or transplant device of claim 9 further comprising a plurality of cells.

17. The tissue scaffold or transplant device of claim 16, wherein said cells are selected from the group consisting of autologous cells, mesenchymal or embryonic stems cells, progenitor cells, and primary cells.

18. A system for storing a polypeptide growth factor for use in tissue repair and engineering applications, comprising:
a scaffold comprising a carrier substrate capable of being covalently cross-linked to the polypeptide growth factor by a transglutaminase; and
a transglutaminase for covalently cross-linking the polypeptide growth factor to the scaffold,
wherein upon cross-linking, said polypeptide growth factor becomes inactivated.

19. The system of claim 18 wherein said polypeptide growth factor is selected from the group consisting of BMP10, BMP15, BMP2, BMP3, BMP4, BMP5, BMP6, BMP7, BMP8A, BMP8B, TGFB1, TGFB2, TGFB3, GDF1, GDF10, GDF11, GDF15, GDF2, GDF3, GDF3A, GDF5, GDF6, GDF7, GDF8, GDF9, GDNF, INHA, INHBA, INHBB, INHBC, INHBE, LEFTY1, LEFTY2, MSTN, NODAL, NRTN, PSPN, AMH, ARTN, FOP, PDGF, VEGF, IGF, and NGF.

20. The system of claim 18, wherein said polypeptide growth factor is a bone morphogenetic protein.

21. The system of claim 18, wherein said polypeptide growth factors are BMP-2.

22. The system of claim 18, wherein said carrier substrate comprises a peptide having an exposed lysine or glutamine residue.

23. The system of claim 18, wherein said carrier substrate is one selected from collagen, gelatin, albumin, fibrin, fibrinogen, laminin, fibronectin, vitronectin or a synthetic peptide containing an exposed lysine or glutamine.

24. The system of claim 18, wherein said transglutaminase is a bacterial transglutaminase.

25. A method for delivering a polypeptide growth factor in a controlled-release manner, comprising:
cross-linking said polypeptide growth factor to a carrier substrate using a transglutaminase to form a storage or delivery vehicle loaded with said polypeptide growth factor, wherein upon cross-linking, said growth factor becomes inactivated; and
introducing said storage or delivery vehicle to a target site for controlled-release of the polypeptide growth factor by an activating agent.

26. The method of claim 25, wherein said polypeptide growth factor is selected from the group consisting of BMP10, BMP15, BMP2, BMP3, BMP4, BMP5, BMP6, BMP7, BMP8A, BMP8B, TGFB1, TGFB2, TGFB3, GDF1, GDF10, GDF11, GDF15, GDF2, GDF3, GDF3A, GDF5, GDF6, GDF7, GDF8, GDF9, GDNF, INHA, INHBA, INHBB, INHBC, INHBE, LEFTY1, LEFTY2, MSTN, NODAL, NRTN, PSPN, AMH, ARTN, FGF, PDGF, VEGF, IGF, and NGF.

27. The method of claim 25, wherein said polypeptide growth factor is selected from a bone morphogenetic protein.

28. The method of claim 25, wherein said polypeptide growth factor is BMP-2.

29. The method of claim 25, wherein said carrier substrate comprises a peptide having an exposed lysine or glutamine.

30. The method of claim 25, wherein said carrier substrate is one selected from collagen, gelatin, albumin, fibrin, fibrinogen, laminin, fibronectin, vitronectin or a synthetic peptide containing an exposed lysine or glutamine.

31. The method of claim 25, wherein said transglutaminase is a bacterial transglutaminase.

32. The method of claim 25, further comprising adding an exogenous activating agent, wherein said exogenous activating agent is a protease capable of enzymatically releasing the polypeptide growth factors from the substrate, thereby activating said growth factors.

33. The method of claim 32, wherein said activating agent is a bacterial protease.

34. The method of claim 32, wherein said activating agent is one selected from the group consisting of the group consisting of pronase, trypsin, chymopapain, chymotrypsin, papain, collagenase, plasmin, pepsin, elastase, MMP1, MMP2, MMP3, MMP8, MMP9, MMP10, MMP13, MMP14, and MMP18.

35. The method of claim 25, further comprising the step of including a plurality of cells in the storage or delivery vehicle, wherein said cells are capable of releasing an activating agent to release and activate the polypeptide growth factors, said activating agent is a metalloproteinase.

36. A method for storing a polypeptide growth factor, comprising:
cross-linking the polypeptide growth factor to a carrier substrate using a transglutaminase,
wherein upon being cross-linked to the substrate, the polypeptide growth factor becomes inactivated.

37. The method of claim 36, wherein:
said polypeptide growth factors are selected from the group consisting of BMP 10, BMP15, BMP2, BMP3, BMP4, BMP5, BMP6, BMP7, BMP8A, BMP8B, TGFB1, TGFB2, TGFB3, GDF1, GDF10, GDF11, GDF15, GDF2, GDF3, GDF3A, GDF5, GDF6, GDF7, GDF8, GDF9, GDNF, INHA, INHBA, INHBB, INHBC, INHBE, LEFTY1, LEFTY2, MSTN, NODAL, NRTN, PSPN, AMH, ARTN, FGF, PDGF, VEGF, IGF, and NGF;
said carrier substrate is selected from collagen, gelatin, or BSA; and
said ttansglutaminase is a bacterial transglutaminase.

38. A method for fabricating a tissue transplant device, comprising:
cross-linking a polypeptide growth factor to a scaffold comprising a carrier substrate using a transglutaminas,
wherein upon cross-linking, said polypeptide growth factor becomes inactivated.

39. The method of claim 38, wherein said polypeptide growth factor is one selected from the group consisting of BMP10, BMP15, BMP2, BMP3, BMP4, BMP5, BMP6, BMP7, BMP8A, BMP8B, TGFB1, TGFB2, TGFB3, GDF1, GDF10, GDF11, GDF15, GDF2, GDF3, GDF3A, GDF5, GDF6, GDF7, GDF8, GDF9, GDNF, INHA, INHBA, INHBB, INHBC, INHBE, LEFTY1, LEFTY2, MSTN, NODAL, NRTN, PSPN, AMH, ARTN, FGF, PDGF, VEGF, IGF, and NGF.

40. The method of claim 38, wherein said polypeptide growth factor is BMP-2.

41. The method of claim 38, wherein said carrier substrate is one having an exposed lysine or glutamine.

42. The method of claim 38, wherein said carrier substrate is selected from collagen, gelatin, albumin, fibrin, fibrinogen, laminin, fibronectin, vitronectin or a synthetic peptide containing an exposed lysine or glutamine.

43. The method of claim 38 further comprising the step of adding a plurality of cells to the scaffold.

44. The method of claim 43, wherein said cells are selected from the group consisting of autologous cells, mesenchymal or embryonic stems cells, progenitor cells, and primary cells.

45. The method of claim 43, further comprising the step of adding an activating agent to the scaffold, wherein said activating agent is one selected from the group the group consisting of pronase, trypsin, chymopapain, chymotrypsin, papain, collagenase, plasmin, pepsin, elastase, MMP1, MMP2, MMP3, MMP8, MMP9, MMP10, MMP13, MMP14, and MMP18.

46. A method for tissue repairing or engineering, comprising:
placing a scaffold at a site in need of tissue repairing or remodeling, wherein said scaffold comprises a polypeptide growth factor covalently cross-linked to a substrate by a transglutaminase, said polypeptide growth factor is in an inactivated state.

47. The method of claim 46, wherein said polypeptide growth factor is selected from the group consisting of BMP10, BMP15, BMP2, BMP3, BMP4, BMP5, BMP6, BMP7, BMP8A, BMP8B, TGFB1, TGFB2,TGFB3, GDF1, GDF10, GDF11, GDF15, GDF2, GDF3, GDF3A, GDF5, GDF6, GDF7, GDF8, GDF9, GDNF, INHA, INHBA, INHBB, INHBC, INHBE, LEFTY1, LEFTY2, MSTN, NODAL, NRTN, PSPN, AMH, ARTN, FGF, PDGF, VEGF, IGF, and NGF.

48. The method of claim 46, wherein said polypeptide growth factor is BMP-2.

49. The method of claim 46, wherein said substrate is gelatin.

50. The method of claim 46, further comprising the step of adding an activating agent to said site after said scaffold is placed at the site, wherein said activating agent is one selected from the group consisting of pronase, trypsin, chymopapain, chymotrypsin, papain, collagenase, plasmin, pepsin, elastase, MMP1, MMP2, MMP3, MMP8, MMP9, MMP10, MMP13, MMP14, and MMP18.
